(19) United States
(12) Patent Application Publication
(10) Pub. No.: US 2026/0256600 A1
(43) Pub. Date: Sep. 3, 2026

(54) REINFORCED PROSTHETIC DEVICE

(71) Applicant: QUORUM ORTHOPEDICS, INC., Windsor, CO (US)

(72) Inventors: Joe Johnson, Windsor, CO (US); John Fleischmann, Windsor, CO (US)

(21) Appl. No.: 19/155,552

(22) PCT Filed: Feb. 12, 2024
(86) PCT No.: PCT/US2024/015403

§ 371 Date: Aug. 11, 2025

Related U.S. Application Data
(60) Provisional application No. 63/444,670, filed on Feb. 10, 2023.

(51) Int. Cl. A61F 2/50

(52) U.S. Cl. CPC A61F 2/50; A61F 2002/5096

(57) ABSTRACT

The present invention is directed to a novel 3D printed prosthetic sockets having one or more internally positioned support struts configured to counter the structural instability at the distal ends of the socket and prevent undesired sheering forces that can result structural failure of the socket.

### CROSS REFERENCE TO RELATED APPLICATIONS

This Patent Cooperation Treaty (PCT) claims the benefit of and priority to U.S. Provisional Application No. 63/444,670, filed Feb. 10, 2023. The entire specification and figures of this related US Application is hereby incorporated, in its entirety by reference.

### TECHNICAL FIELD

The present invention is directed to systems, methods and devices to reinforce a prosthetic socket frame, and in particular the distal portion of a 3D printed socket frame.

### BACKGROUND

Traditional prosthetic devices typically operate by securing the residual limb into a rigid or semi-rigid socket. A socket may commonly refer to the portion of a prosthesis that fits around and secures a residual limb, and to which prosthetic components, such as a foot, are attached. Traditional prosthetic devices, such as sockets, are generally designed to stabilize the skeletal components of the residual limb and allow minimal relative movement between the socket and the residual limb. To effectively support the residual limb and allow for the efficient transfer of mechanical loads from the residual limb to the ground, the prosthetic sockets are designed to provide a sufficient support to secure the residual limb within the socket, while at the same time allowing sufficient flexibility to allow for circulation and account for other physiological, temporal or environmental changes that may affect the shape and/or volume of the residual limb.

However, with the development of prosthetic devices manufactured using 3D printed technologies, certain structural limitations have been identified that required novel developments in prosthetic design. For example, as noted below, Applicants have demonstrated that structural failure of 3D printed sockets most commonly occurs at the distal end of the prosthetic socket below the coupler attachments. In particular, this structural failure can result in a total shearing of the distal end parallel to the bottom surface of these internal couplers, most commonly t-nuts used to secure a lower limb, or other attachment to the socket frame. As such, there is a need for novel 3D printed prosthetic socket designs to counter the structural instability at the distal ends of the socket and prevent undesired sheering forces that can result in the structural failure of the socket.

### SUMMARY OF THE INVENTION

In one aspect the inventive technology includes is directed to systems, methods and devices to reinforce a prosthetic socket frame, and in particular the distal portion of a 3D printed socket frame using. In this aspect, a socket frame, and preferably a 3D printed socket frame can be manufactured to include one or more channels positioned at the distal end of the frame. The channels of the invention may be positioned approximately vertical to the shear plane positioned below internally positioned couplers, such as t-nuts, that are further configured to secure a coupler plate. At least one support strut may be inserted into each of the channels so as to be positioned perpendicular to the shear plane. In this configuration, the perpendicular support strut can strengthen the socket frame at the shear plane and increases the maximum load required to create a sheer force mode of failure.

Additional aspects of the invention may become evident based on the specification and figures presented below.

### BRIEF DESCRIPTION OF THE DRAWINGS

FIG. 1: shows a bottom perspective view of a 3D printed socket frame having a plurality of support channels positioned along the outer edge of a distal plate surface in one embodiment thereof;

FIG. 2: shows a side view of a 3D printed socket frame having a plurality of support struts positioned across and approximately perpendicular to the shear plane of the distal end of the socket in one embodiment thereof;

FIG. 3: shows a side view of a 3D printed socket frame having a plurality of couplers positioned adjacent to a corresponding plate aperture and above the shear plane in one embodiment thereof; and

FIG. 4: is a close-up view of the f a 3D printed socket frame having a plurality of support struts positioned across and approximately perpendicular to the shear plane of the distal end of the socket in one embodiment thereof;

### DETAILED DESCRIPTION OF THE INVENTION

In one embodiment the invention may include a prosthetic device (1), which preferably includes a-D printed socket frame (2). In one preferred embodiment, a diagnostic evaluation of a patient in need of a prosthetic device (1) may be performed and the initial three-dimensional shape of the residual limb may be digitally generated. The digital generation of this 3D model may be captured by invasive, or non-invasive diagnostic techniques known in the art. In a preferred embodiment, this 3D model of the residual limb may be digitally captured and communicated to a computer system that may further process the 3D model and generate a digital output for a customized 3D printed socket frame (2) configured to conform to the shape of the 3D model of the residual limb. In this embodiment, the digital output for a customized 3D printed socket frame (2) may be transmitted to a fabrication component or may be outputted into a CAD or other file format for automated mechanical or manual production. In another preferred embodiment, the computer system may upload the 3D model and generate a digital output for a customized 3D printed socket frame (2) configured to conform to the shape of the 3D model of the residual limb that may further be input into a 3D fabrication device, such as a 3D printer. The 3D printing device may execute the 3D model file and fabricate a rigid socket frame (2) and/or inner socket (not showing) configured to conform to the shape of the 3D model of the residual limb. Generally, a rigid socket frame (2) and/or inner socket generated by a rapid 3D printing/prototyping process may be made from a variety of materials, and preferably composites, generally known in the art. Exemplary 3D-printed prosthetic devices (1) and rigid socket frame (2) are described by Johnson et al., in U.S. application Ser. No. 15/930,797, incorporated herein by reference.

In one embodiment, the invention may include a socket frame (2), and preferably a 3D printed rigid socket frame (2) generated by a rapid 3D printing/prototyping, further configured to incorporate additional reinforcing structures at its distal end as generally described below.

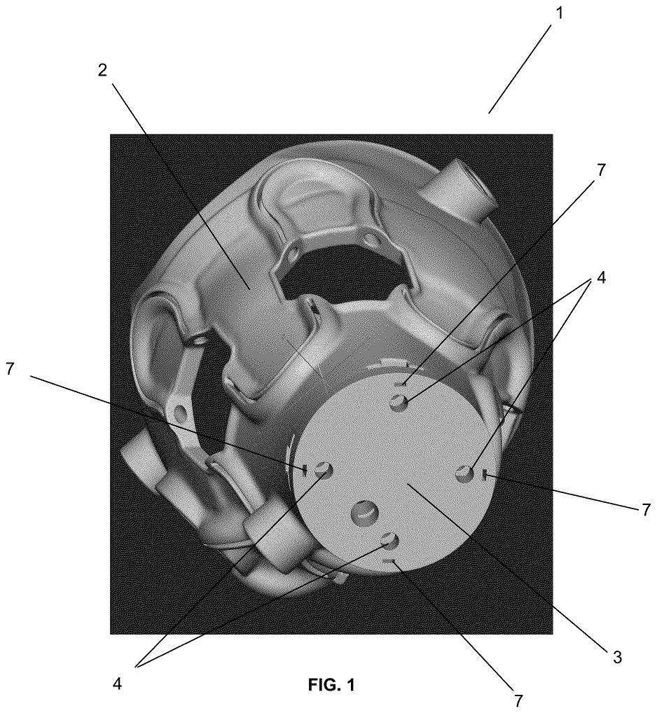

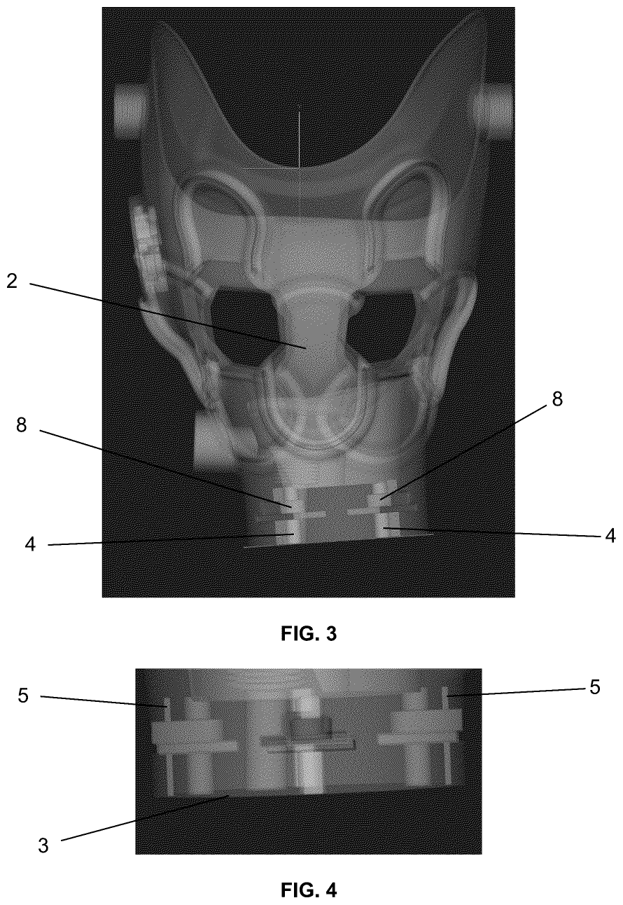

As shown in FIGS. 1 and 3, the socket frame (2) of the invention may be a rigid form configured to accommodate a residual limb (not shown). One exemplary embodiment shown in the figures includes a transfemoral socket frame, or a socket frame (2) configured to accommodate a residual limb wherein the knee joint has been removed and the individual still has part of the femur or thighbone intact. Additional embodiments not specifically shown may also be contemplated, which may include, but not be limited to: a transhumeral socket frame; a transradial socket frame; a transtibial socket frame; a symes socket frame; a hip disarticulation socket frame; a knee disarticulation socket frame; and a wrist disarticulation socket frame and the like.

As noted above, in a preferred embodiment a socket frame (2) may be formed of a rigid material to accommodate and provide a support for a residual limb. In one preferred embodiment, a rigid socket frame (2) may be formed from a variety of materials, including but not limited to: plastic, composites, carbon fiber or even an acrylic laminate socket frame with a stiffening component such as carbon fiber and/or para-aremid synthetic fiber.

Generally referring to FIG. 1, socket frame (2) of the invention may include a plate surface (3). In the embodiment shown in the figures, the plate surface (3) of the invention can be configured as an integral portion of a 3D printed socket frame (2) and is further configured to secure additional prosthetic components such as artificial limbs, mechanical devices, as well as shock absorbers and the like. As shown in FIG. 1, the plate surface (3) of the invention may further include one, or preferably a plurality of plate apertures (4) extending into the distal portion of the socket frame (2). The plate apertures (4) may be positioned adjacent to a coupler (8), such as a t-nut also positioned internally at the distal portion of the socket frame (2). In this configuration, a fastener (not shown), such as a screws or bolt can be positioned through a metal plate (not shown), and aligned with the plate apertures (4) such that the fasteners are inserted into the apertures and engage the coupler (8), which may preferably include a t-nut, thereby attaching a metal plate to the socket frame (2) Then the metal plate, sometimes referred to as a 4-hole plate may include one or more attachment points to secure additional prosthetic components such as a shock absorber or pylon or directly to a foot or ankle in this example.

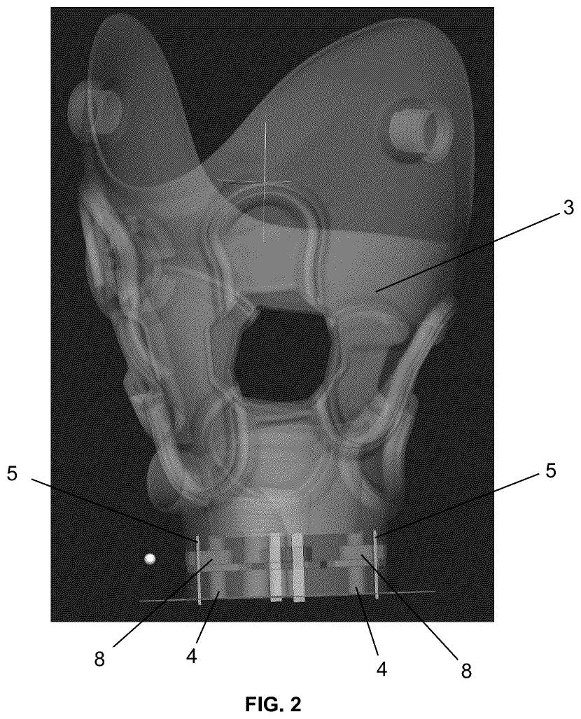

The invention may further include one or more support channels (7). In the preferred embodiment shown in the figures, the plate surface (3) of the invention may include one, or preferably a plurality of support channels (7). These support channels (7) may be integral to the socket frame (2), and may further traverse from the plate surface (3) into the socket frame (2). As shown in FIG. 1, the support channels (7) of the invention may be positioned adjacent to the plate apertures (4), and are preferably positioned between the edge or the plate surface (3) and the plate apertures (4). Notably, as shown in FIG. 2, the support channels (7) of the invention traverse the shear plane (9).

The invention may further include one or more support strut (5). As shown in FIGS. 2 and 4, a support strut (5) may include a metal or composite pin or rod that is inserted into a support channels (7). In this configuration, the support strut (5) traverses across the shear plane (9) in a perpendicular orientation. In the preferred configuration shown in the figures, the support strut (5) are positioned external and adjacent to a coupler (8) and may further include one or more paired support strut (5) positioned in an opposing arrangement around the outer rim of the plate surface (3). In this embodiment, each plate aperture (4) may include a corresponding support strut (5) positioned in an approximately parallel orientation. In this configuration, the support strut (5) strengthens the distal end of the socket frame (2) at the shear plane (9) and increases the maximum load required to create a sheer force mode of failure.

In additional preferred embodiments, the support strut (5) of the invention can be further secured in a corresponding support channels (7) by a lock, which may include a detachable lock (not shown) or an adhesive, such as an epoxy and the like.

Naturally, all embodiments discussed herein are merely illustrative and should not be construed to limit the scope of the inventive technology consistent with the broader inventive principles disclosed. As may be easily understood from the foregoing, the basic concepts of the present inventive technology may be embodied in a variety of ways. It generally involves systems, methods, techniques as well as devices to accomplish a reinforced prosthetic device and the like. In this application, the methods and apparatus for the aforementioned systems are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the statements of invention. As can be easily understood from the foregoing, the basic concepts of the present invention may be embodied in a variety of ways. It involves both techniques as well as devices to accomplish the appropriate system. In this application, the techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention may be described in some instances in method-oriented terminology, each element of the claims corresponds to a device and vice versa. Apparatus claims may not only be included for the device described, but also method or process claims may be included to address the functions the invention and each element performs. Neither the description nor the terminology is intended to limit the scope of the claims that will be included in any subsequent patent application.

It should also be understood that a variety of changes may be made without departing from the essence of the invention. Such changes are also implicitly included in the description. They still fall within the scope of this invention. A broad disclosure encompassing the explicit embodiment(s) shown, the great variety of implicit alternative embodiments, and the broad methods or processes and the like are encompassed by this disclosure and may be relied upon when drafting any claims. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of the invention both independently and as an overall system.

Further, each of the various elements of the invention and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “support” should be understood to encompass disclosure of the act of “supporting”—whether explicitly discussed or not—and, conversely, were there effectively disclosure of the act of “supporting”, such a disclosure should be understood to encompass disclosure of a “supporting method and/or technique, and/or device” and even a “means for supporting.” Such changes and alternative terms are to be understood to be explicitly included in the description.

Thus, the applicant(s) should be understood to have support to claim and make a statement of invention to at least: i) each of the methods and/or apparatus for providing a reinforced prosthetic device as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) each system, method, and element shown or described as now applied to any specific field or devices mentioned, x) methods and apparatuses substantially as described hereinbefore and with reference to any of the accompanying examples, xi) the various combinations and permutations of each of the elements disclosed, xii) each potentially dependent claim or concept as a dependency on each and every one of the independent claims or concepts presented, and xiii) all inventions described herein.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479F. 3d 1313(Fed. Cir 2007), or the like are expressly not intended in this or any subsequent related matter. In addition, support should be understood to exist to the degree required under new matter laws including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible. It should be understood that this application also provides support for any combination of elements in the claims and even incorporates any desired proper antecedent basis for certain claim combinations such as with combinations of method, apparatus, process, and the like claims.

Any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon. The inventive subject matter is to include, but certainly not be limited as, a system substantially as herein described with reference to any one or more of the Figures and Description (including the following: for example, the process according to any claims and further comprising any of the steps as shown in any Figures, separately, in any combination or permutation).

1. A reinforced prosthetic assembly comprising:
a socket frame having a support channel positioned at the distal end of the frame;
at least one support strut positioned within said support channel, wherein said support strut traverses a shear plane, thereby reinforcing the distal portion of said socket frame.

2. The prosthetic assembly of claim 1, wherein said socket frame comprises a 3D printed socket frame.

3. The prosthetic assembly of claim 2, wherein said 3D printed socket frame is selected from: a transfemoral socket frame, a transhumeral socket frame, a transradial socket frame, a transtibial socket frame, a symes socket frame, a hip disarticulation socket frame, a knee disarticulation socket frame, and a wrist disarticulation socket frame.

4. The prosthetic assembly of claim 1, wherein said support channel is positioned adjacent to a coupler.

5. The prosthetic assembly of claim 4, wherein said coupler comprises a T-nut.

6. The prosthetic assembly of claim 1, wherein said socket frame further comprises a plate surface.

7. The prosthetic assembly of claim 7, wherein said support channel is positioned adjacent to a plate aperture on said plate surface.

8. The prosthetic assembly of claim 1, wherein said support strut is selected from: a metal support strut, or a composite support strut, or a carbon-fiber support strut.

9. The prosthetic assembly of claim 1, wherein said sheer surface is positioned at the distal end of said socket frame below a coupler.

10. The prosthetic assembly of claim 1, wherein said support strut is secured in said support channel by a lock.

11. The prosthetic assembly of claim 1, wherein said support strut is secured in said support channel by an adhesive.

12. The prosthetic assembly of claim 1, wherein said support channel comprises a plurality of support channels positioned at the distal end of the frame.

13. The prosthetic assembly of claim 12, wherein said plurality of support channels comprise four support channels positioned in an opposing paired configuration.

14. The prosthetic assembly of claim 13, wherein said four support channels are equidistant from one another.

15. The prosthetic assembly of claim 12, wherein said plurality of support channels are equidistant from one another and are positioned between a corresponding plate aperture and the edge of plate surface.

16. The prosthetic assembly of claim 11, wherein said support strut comprises a plurality of support struts.

17. The prosthetic assembly of claim 16, wherein said plurality of support struts comprises at least one support strut positioned within a corresponding support channel.

18. A reinforced 3D printed prosthetic device comprising:
a 3D printed socket frame having a support channel positioned on a plate surface at the distal end of the frame;
at least one support strut positioned within said support channel, wherein said support strut traverses a shear plane, thereby reinforcing the distal portion of said socket frame.

19. The 3D printed prosthetic device of claim 18, wherein said 3D printed socket frame is selected from: a transfemoral socket frame, a transhumeral socket frame, a transradial socket frame, a transtibial socket frame, a symes socket frame, a hip disarticulation socket frame, a knee disarticulation socket frame, and a wrist disarticulation socket frame.

20. The 3D printed prosthetic device of claim 18, wherein said support strut is selected from: a metal support strut, a composite support strut, or a carbon-fiber support strut.
